(19) United States
(12) Patent Application Publication
(10) Pub. No.: US 2024/0180426 A1
(43) Pub. Date: Jun. 6, 2024

(54) MODULATED PHYSIOLOGICAL SENSOR

(71) Applicant: MASIMO CORPORATION, Irvine, CA (US)

(72) Inventors: Marcelo Lamego, Cupertino, CA (US); Cristiano Dalvi, Lake Forest, CA (US); Hung Vo, Fountain Valley, CA (US)

(21) Appl. No.: 18/537,615

(22) Filed: Dec. 12, 2023

Related U.S. Application Data
(63) Continuation of application No. 17/208,501, filed on Mar. 22, 2021, now Pat. No. 11,877,824.
Application No. 17/208,501 is a continuation of application No. 15/729,240, filed on Oct. 10, 2017, now Pat. No. 10,952,614.
Application No. 15/729,240 is a continuation of application No. 13/584,447, filed on Aug. 13, 2012, now Pat. No. 9,782,077.
(60) Provisional application No. 61/524,744, filed on Aug. 17, 2011.
(60) Provisional application No. 61/639,985, filed on Apr. 29, 2012.

(51) Int. Cl. A61B 5/00

(52) U.S. Cl. CPC A61B 5/0051; A61B 5/4884; A61B 5/6843; A61B 2562/0219

(57) ABSTRACT

A modulated physiological sensor is a noninvasive device responsive to a physiological reaction of a living being to an internal or external perturbation that propagates to a skin surface area. The modulated physiological sensor has a detector configured to generate a signal responsive to the physiological reaction. A modulator varies the coupling of the detector to the skin so as to at least intermittently maximize the detector signal. A monitor controls the modulator and receives an effectively amplified detector signal, which is processed to calculate a physiological parameter indicative of the physiological reaction.

### CROSS-REFERENCE TO RELATED APPLICATIONS

The present application is a continuation of U.S. patent application Ser. No. 17/208,501, filed Oct. 10, 2017, which, is a continuation of U.S. patent application Ser. No. 15/729,240, filed Mar. 22, 2021, which is a continuation of U.S. patent application Ser. No. 13/584,447, filed Aug. 13, 2012, which claims priority benefit under 35 U.S.C. § 119(e) to U.S. Provisional Patent Application Ser. No. 61/524,744, filed Aug. 17, 2011, titled Modulating Physiological Sensor and U.S. Provisional Patent Application Ser. No. 61/639,985, filed Apr. 29, 2012, titled Modulated Physiological Sensor, both provisional applications hereby incorporated in their entirety by reference herein.

### BACKGROUND OF THE INVENTION

From a physiological perspective, the human body comprises a set of interacting systems, each having specific functions and purposes. These systems maintain the body's internal stability by coordinating the response of its parts to any situation or stimulus that would tend to disturb its normal condition or function. The nervous system includes the central nervous system and the peripheral nervous system. The central nervous system is the brain and the spinal cord. The musculoskeletal system includes the skeleton and attached muscles and includes bones, ligaments, tendons, and cartilage. The circulatory system includes the heart and blood vessels, including arteries, veins and capillaries. The respiratory system includes the nose, trachea and lungs. The gastrointestinal system includes the mouth, esophagus, stomach, intestines, liver, pancreas and gallbladder. The integumentary system includes the skin, hair, nails, sweat glands and sebaceous glands. The urinary system includes the kidneys and bladder. The immune system includes white blood cells, thymus and lymph nodes. The endocrine system includes the pituitary, thyroid, adrenal and parathyroid glands.

Various sensors may be applied for analyzing and measuring the processes occurring in the above-cited physiological systems and for generating physiological parameters indicative of health or wellness as a result. As one example, a pulse oximetry sensor generates a blood-volume plethysmograph waveform from which oxygen saturation of arterial blood and pulse rate may be determined, among other parameters. As another example, an acoustic sensor may be used to detect airflow sounds in the lungs, bronchia or trachea, which are indicative of respiration rate.

### SUMMARY OF THE INVENTION

The physiological systems cited above maintain the stability, balance and equilibrium of a living being. Modulation may be advantageously used to accentuate detection of processes occurring within these physiological systems. An example of natural modulation is tissue vibration in the trachea due to the inflow and outflow of air between the lungs and the nose and mouth. This vibration creates sound waves at a higher frequency than the underlying respiration. An acoustic sensor utilizing a piezoelectric device attached to the neck is capable of detecting these sound waves and outputting a modulated sound wave envelope that can be demodulated so as to derive respiration rate. An acoustic respiration rate sensor and corresponding sensor processor is described in U.S. patent application Ser. No. 12/904,789, filed Oct. 14, 2010, titled Acoustic Respiratory Monitoring Systems and Methods, assigned to Masimo Corporation, Irvine, CA (“Masimo”) and incorporated by reference herein.

Another example of natural modulation is pulsatile arterial blood flow at a peripheral tissue site, such as a fingertip, resulting from pressure waves generated by the heart. An optical sensor generates a plethysmograph waveform responding to changes in a light absorption due to the pulsatile blood flow so as to measure blood composition, such as hemoglobin constituents. This plethysmograph also modulates a respiration envelope that can be demodulated so as to derive respiration rate.

An example of artificial modulation is a physiological sensor having an accelerometer and a vibration element mounted on a substrate so that the vibration element is in mechanical communications with the accelerometer. An interface communicates at least one axis of the accelerometer signal to a monitor. The substrate is attached to the skin surface of a living being, and the vibration element is activated so as to modulate the skin surface coupling at a modulation frequency. In an embodiment, an artificially-modulated sensor is responsive to respiratory-induced movements at the skin surface.

One aspect of a modulated physiological sensor is a noninvasive sensor responsive to a physiological reaction of a living being to an internal or external perturbation that propagates to a surface area of the living being. The modulated physiological sensor has a detector configured to communicate with a surface area of a living being so as to generate a signal responsive to a physiological reaction of the living being to the perturbation. A modulator varies the coupling of the detector to the surface area so as to at least intermittently maximize the detector signal. A monitor controls the modulator and receives a detector signal so as to calculate a physiological parameter indicative of a physiological state of the living being.

In various embodiments, the modulator is a vibration element that mechanically accentuates the coupling of the detector to the surface area. A substrate co-mounts the detector and the vibration element. An attachment releasably affixes the substrate, detector and vibration element to the surface area. In an embodiment, the detector is an accelerometer and the vibration element is a coin motor. The substrate is a circuit board that mechanically mounts and electrically interconnects the accelerometer and coin motor. The attachment is a tape having a sticky side that attaches to the surface area and a housing side that encloses the circuit board.

Another aspect of a modulated physiological sensor is a sensing method the provides a detector responsive to a physiological wave generated within a living being that propagates to a skin surface and couples the detector to the skin surface. The detector coupling is modulated so as to generate a modulated detector output indicative of the physiological wave. The detector signal is demodulated so as to derive a physiological signal, and a physiological parameter is determined from the physiological signal. In various embodiments, the modulation is vibration of the detector by co-mounting the detector and a vibration element. The detector and the vibration element may be co-mounted to a common substrate, which is attached to the skin surface. A second detector and a second vibration element may be mounted to the common substrate and isolated from the combination detector and vibration element.

A further aspect of a modulated physiological sensor is a detector means for responding to physiological propagations reaching a skin surface of a living being and a modulator means for varying the coupling of the detector means to the skin surface. A monitor demodulates a sensor signal from the detector means so as to analyze the physiological propagations and generate a physiological parameter output. In various embodiments, a substrate means mounts the detector means and the modulator means and an attachment means secures the substrate to the skin surface. A control signal from the monitor sets a frequency of the modulator means above a low frequency cutoff of the detector means. In an embodiment, the modulator means is a vibration element, the detector means is multiple detectors, the modulator means is multiple vibration elements and the substrate means incorporates at least one isolation element so as to isolate detector and vibration element pairs. In an embodiment, the vibration element remotely modulates the detector via an acoustic wave.

### BRIEF DESCRIPTION OF THE DRAWINGS

FIG. 1 is a general block diagram of a modulated physiological sensor in communications with the physiological systems of a living being;

FIG. 2 is general block diagram of a modulated physiological sensor embodiment;

FIGS. 3A-D are amplitude vs. time and corresponding amplitude vs. frequency graphs of a physiological reaction and a corresponding modulated and detected reaction;

FIG. 4 is a general block diagram of a vibration-modulated physiological sensor embodiment;

FIG. 5 is a general block diagram of a multi-element, vibration-modulated sensor embodiment;

FIGS. 6A-F are side views of various modulated physiological sensor embodiments;

FIG. 7 is a general block diagram of a vibration-accelerometer physiological sensor embodiment;

FIG. 8 is a detailed block diagram of a vibration-accelerometer physiological sensor embodiment;

FIG. 9A-B are assembled and exploded perspective views, respectively, of a vibration-accelerometer physiological sensor embodiment; and

FIG. 10 is a graph of a vibration-accelerometer physiological sensor output versus time illustrating three-axis of respiration envelopes with the vibration turned on and off.

### DETAILED DESCRIPTION OF THE PREFERRED EMBODIMENTS

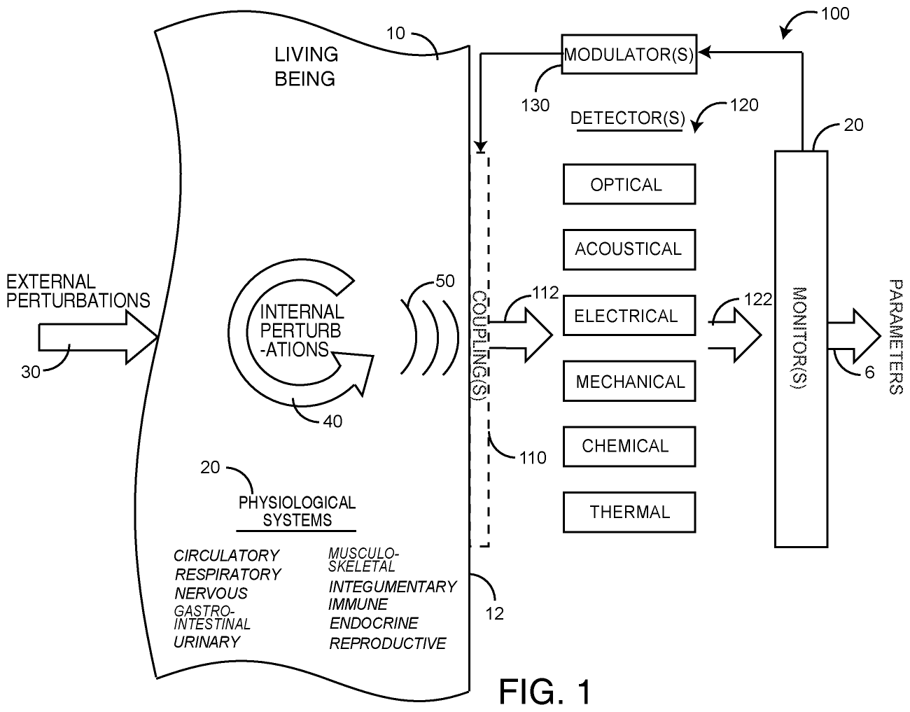

FIG. 1 generally illustrates a modulated physiological sensor 100 in communications with the physiological systems 20 of a living being 10. Physiological reactions 50 to external 30 or internal 40 perturbations propagate to the body surface 12 and are coupled 110 to one or more detectors 120. These physiological reactions 50 are indicative of states and processes of the physiological systems 20. The detectors 120 are responsive to coupled physiological reactions 112 so as to generate detector outputs 122. One or more monitors 20 are responsive to the detector outputs 122 so as to compute physiological parameters 6 that quantify the states and processes of the physiological systems 20. The coupling(s) 110 is advantageously modulated 130 under control of the monitor(s) 20 so as to accentuate detection of the physiological reactions 50, as described in further detail below.

As shown in FIG. 1, detectors 120 include any device that is responsive to the coupled physiological reactions 112 such as optical, acoustical, electrical, mechanical, chemical and thermal mechanisms, to name a few. The detector outputs 122 may include blood photo-plethysmographs, ECG, EEG and body sound waveforms; indications of skin color, temperature, movement or pressure; and chemical responses and measurements of moisture, breath, sweat or odors, to name a few. The monitor(s) 20 may include any or all devices or combinations of devices that are responsive to the detector outputs 122 alone or in combination so as to calculate or otherwise derive physiological parameters 6 that measure, graph, quantify or otherwise indicate one or more aspects of the physiological systems 20 and corresponding states and processes corresponding to the physiological reactions 50. Parameter examples include circulatory system measurements such as oxygen saturation, heart rate, blood glucose and blood pressure; and respiratory system measurements such as respiration rate and volume, to name but a few. Parameters 6 can also include indications of specific abnormal physiological conditions such as sleep apnea, anemia and hypoglycemia, to name a few.

Also shown in FIG. 1, external perturbations 30 may be natural, such as changes to a person's physical environment including temperature, pressure, light and sound, for example. External perturbations 30 also may be artificial, such as the mechanical pressure induced by a respirator for breathing assistance or by a pulser on a fingertip for measuring venous oxygen saturation as examples. Internal perturbations 40 include normal and abnormal functioning and interactions of various physiological systems 20, including circulatory and respiratory functions, to name a few. Internal perturbations 40 may also be artificial, such as due to a pacemaker or other implanted device. Physiological reactions 50 resulting from external perturbations 30 or internal perturbations 40 include, as examples, a body surface expansion or contraction due to, say, lung inflation/deflation; an acoustic wave arriving from within the body to the body surface due to a heart beat or bowel sound; or a transverse wave traveling along the body surface due to a muscle spasm. In general a physiological reaction 50 may be an optical, acoustical, electrical, mechanical, chemical or thermal impulse, wave or other variation or change. Further, external perturbations 30 or internal perturbations 40 need not be the same type or kind (e.g. optical, acoustical, electrical, mechanical, chemical or thermal) as the corresponding physiological reaction 50 or the detector element 120 responsive to the physiological reaction 50. For example, an injection (external chemical perturbation) may trigger a heart arrhythmia that results in an acoustic and a mechanical wave (physiological reaction) that propagates to the skin surface and is detected by an acoustical or mechanical sensor, or both. Further, the heart arrhythmia may result in an arterial pulse abnormality that changes the optical characteristics of a tissue site as measured by an optical sensor attached to the tissue site.

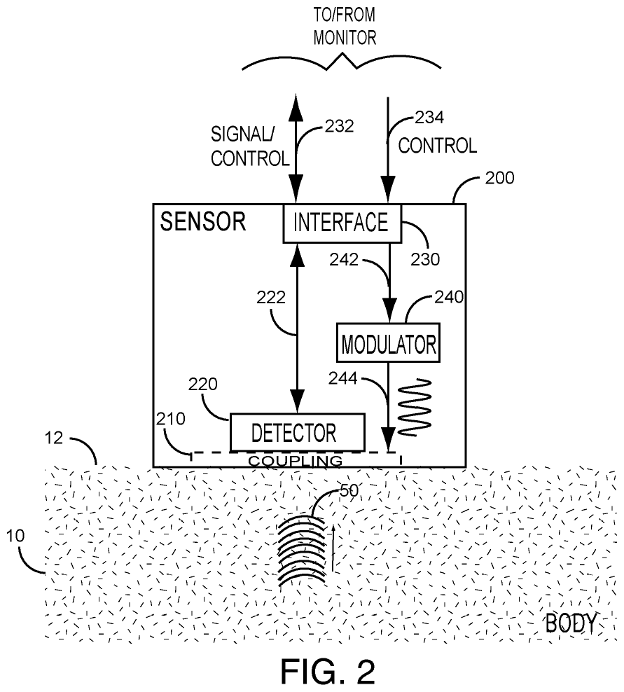

FIG. 2 illustrates a modulated physiological sensor 200 embodiment that attaches to a body surface 12 and is configured to respond to physiological reactions 50, as described above. The sensor 200 has a coupling 210, a detector 220, an interface 230 and a modulator 240. A monitor (not shown) outputs controls 232, 234 to the sensor 200 and receives signals 232 from the sensor 200. The interface 230 communicates detector signals 222 to the monitor in response to drive controls 222 to the detector 220. The interface 230 also communicates a modulator control 242 to the modulator 240. The modulator 240 responds to the modulator control 242 so as to generate a modulation 244 to the coupling 210.

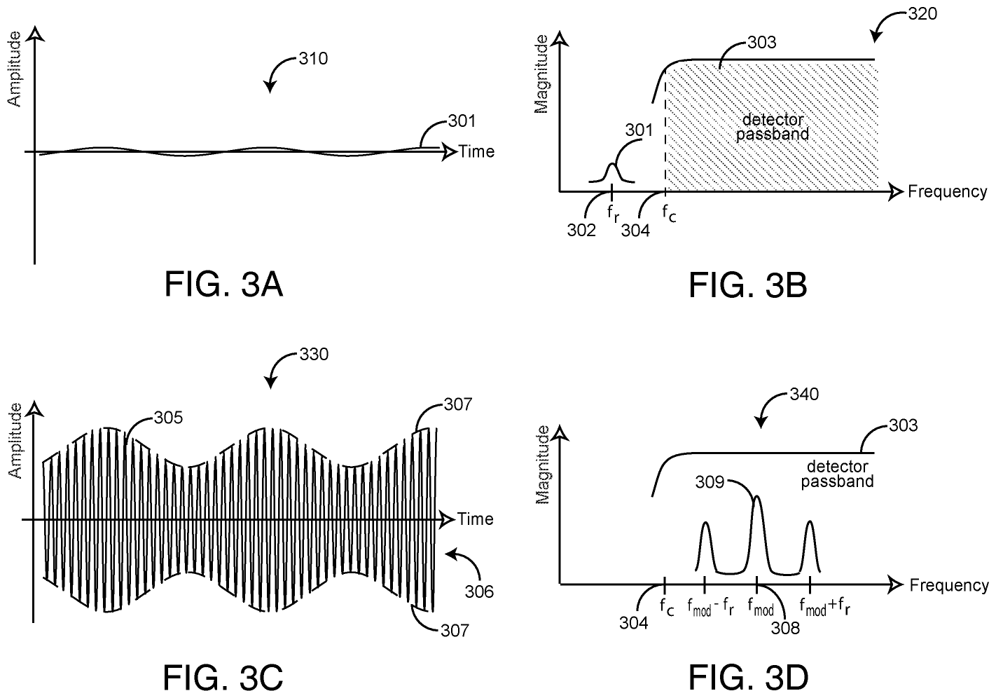

As shown in FIG. 2, the modulator 240 varies the coupling 210 of the detector 220 to the body surface 12 and hence to the physiological reaction 50. In particular, the body surface 12 of a person, including skin and underlying tissues, varies by individual and, indeed, by location on a particular individual. These variations are in shape, texture, color and elasticity to name a few. As such, a fixed coupling is unlikely to provide an optimum body surface/detector interface. Indeed efficient and effective body surface/detector coupling is an issue for most if not all physiological sensors. For example, common ECG electrodes require a conductive gel so as to effectively couple to a skin surface. The modulator 240 advantageously continuously varies the detector coupling 210 to the skin surface across a range of contact forces at the skin/sensor interface. For an electrical detector, say, this varied coupling alters the detector electrical resistance at the skin surface over a range of values. For a mechanical detector, the varied coupling alters the mechanical impedance of the detector at the skin surface over a range of values. For an acoustic detector, for example, the varied coupling alters the acoustical impedance of the detector at the skin surface over a range of values. As a result of this variable detector coupling to the skin surface, the detector has maximal and minimal coupling each modulation cycle. Further, the modulation frequency may be set above any detector low frequency response cutoffs. Accordingly, the modulation advantageously amplifies the detector signal 222, as described in further detail with respect to FIGS. 3A-D, below.

FIGS. 3A-D illustrate a physiological system reaction to perturbations and a corresponding modulated and detected sensing of the reaction. FIG. 3A is an exemplar time domain graph 310 of a relatively low amplitude, low frequency physiological system reaction 301 to some form of internal or external perturbation. FIG. 3B is a corresponding exemplar frequency domain graph 320 of the physiological system reaction 301. The physiological reaction 301 may be difficult to detect due to either a small amplitude signal 301 or a signal frequency f_{f} 302 less than the detector cutoff frequency f_{c} 304, i.e. outside the detector passband 303.

FIG. 3C is an exemplar time domain graph 330 of a modulated detector response 305 to the reaction 301 (FIG. 3A) described above. The response 305 has a modulation 306 and an envelope 307. In particular, the physiological sensor 200 (FIG. 2) has a modulated coupling 210 (FIG. 2) that achieves or approaches a maximal coupling of a detector 220 (FIG. 2) to a body surface 12 (FIG. 2) at least once per modulation cycle, as described with respect to FIG. 2 above. Accordingly, the modulated detector 220 (FIG. 2) accentuates the physiological signal 301 (FIG. 3A) during the maximal coupling and de-accentuates the physiological signal 301 (FIG. 3A) during the minimal coupling. This cyclical accentuation/de-accentuation generates an envelop 307 that is, effectively, an amplification of the physiological reaction 301 (FIG. 3A).

FIG. 3D is an exemplar frequency domain graph 340 of a modulated physiological sensor response 305 (FIG. 3C). In various embodiments, the modulation frequency f_{mod} 308 is set substantially higher than any low frequency cutoff f_{c} 304 of the detector so that the sensor response 305 is well within the detector passband 303 (FIG. 3B).

As described with respect to FIGS. 3A-D, in various embodiments an amplified version of the physiological response 301 (FIG. 3A) is derived from the sensor response 305 (FIG. 3C) by any of various well-known AM demodulation techniques. These include envelope detection with a rectifier or product detection utilizing multiplication by a local oscillator, to name a few.

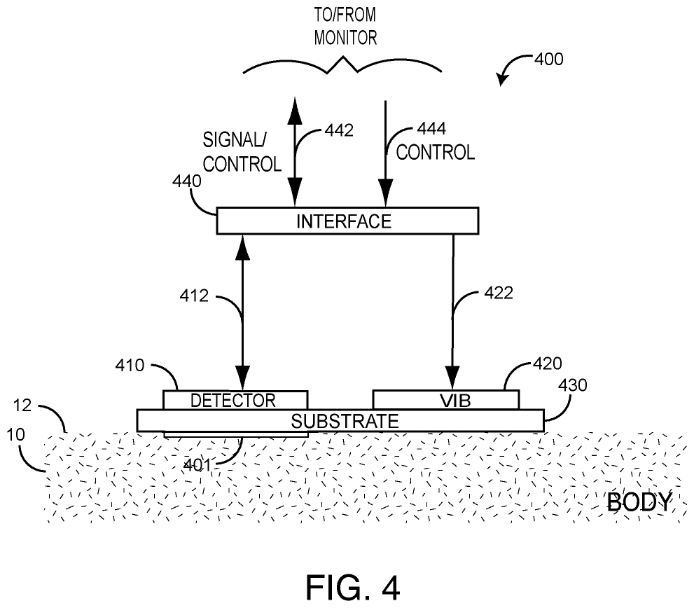

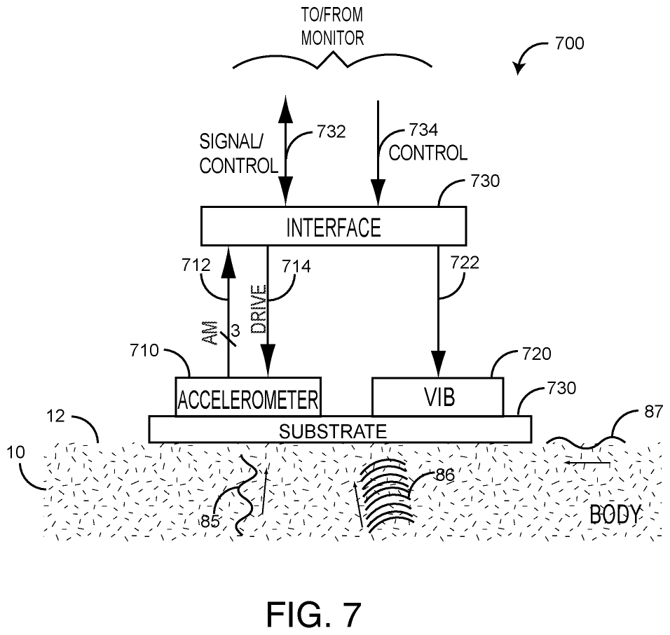

FIG. 4 illustrates a vibration-modulated physiological sensor 400 embodiment. The sensor 400 has a detector 410, a vibration element (“vib”) 420, a substrate 430 and an interface 440 to a monitor. The detector 410 and the vib 420 are both mounted to the substrate 430. In an embodiment, the detector 410 is mounted so as to directly couple 401 to the body surface 12. For example, the detector 410 may be mounted through the substrate 430, as shown. In other embodiments, the detector 410 is attached adjacent the substrate 430. In additional embodiments, the detector 410 may not contact the body surface 12 at all, such as with an accelerometer-based detector described with respect to FIGS. 7-10, below. In an embodiment, the vib 420 is a coin motor, as described with respect to FIGS. 7-10, below. In other embodiments, the vib 420 is any of various off-balance motors, voice coils or similar electro-mechanical devices. In further embodiments, the vib 420 is any mechanical, electromagnetic, piezoelectric, pneumatic, electric, acoustic or magnetic device that vibrates in response to an electrical signal.

As shown in FIG. 4, the detector 410, and hence the coupling 401, is vibration-modulated 420 via the substrate 430. The substrate 430 may be any material that effectively transmits or conducts vibrations from the vib 420 to the detector 401. In an advantageous embodiment, the substrate 430 is a circuit board material that provides mechanical mounts for and supports electrical interconnects between the sensor components.

Also shown in FIG. 4, a monitor (not shown) outputs controls 442, 444 to the sensor 400 and receives signals 442 from the sensor 400. The interface 440 communicates detector signals 412 to the monitor in response to drive controls 412 to the detector 410. The interface 440 also communicates a vibration control 422 to the vib 420. The vib 420 responds to the vibration control 422 so as to generate a modulation to the coupling 401 via the substrate 430. In various embodiments, the detector 410 may be mechanical, such as an accelerometer described with respect to FIGS. 7-10, below. In other embodiments, the detector 410 may be electrical, such as an electrode for sensing ECG or EEG signals; or optical such as a photodiode; or acoustical, such as a piezoelectric device; or thermal, such as a thermopile, pyrometer, thermistor, thermocouple, IR photodiode or temperature diode, to name a few.

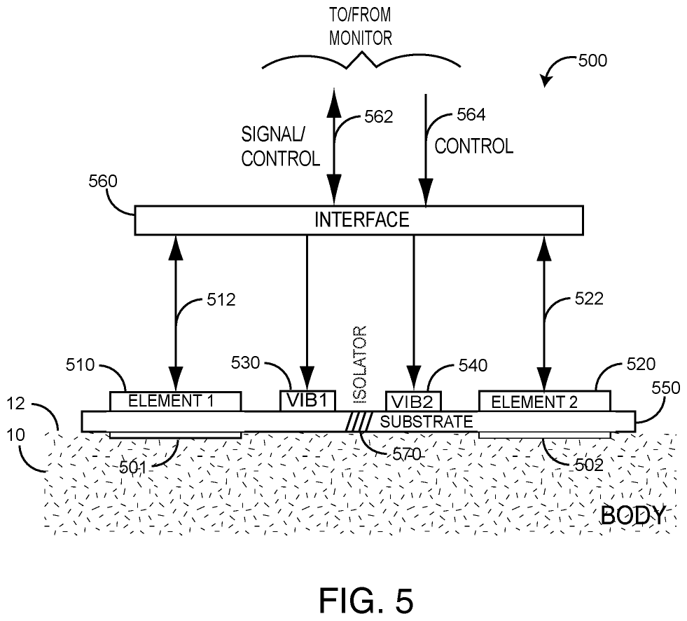

FIG. 5 illustrates a multiple-element, vibration-modulated sensor 500 embodiment having a two or more sensor elements 510, 520, one or more vibration elements (vibs) 530, 540, a substrate 550 and an interface 560 to a monitor. The sensor elements 510, 520 may each be detectors or a combination of one or more detectors and one or more emitters. In an embodiment, the sensor elements 510, 520 are different types of detectors. For example, element1 510 may be mechanical and element2 may be electrical. In an embodiment, the sensor elements 510, 520 may be an emitter and a corresponding detector. For example, element1 510 may be an LED for illuminating a tissue site and element2 520 may be a optical detector, such as a diode or diode array, for receiving the LED illumination after attenuation by the tissue site. Advantageously, multiple elements 510, 520 on a single substrate 550 provide an array of like sensors for increased detection capability or for directional sensing capability, such as determining the source of a body sound as but one example. Advantageously, multiple elements 510, 520 on a single substrate 550 provide an array of different sensors in a single sensor package for simultaneous detection and analyses of multiple types or kinds of physiological responses to the same or different external or internal perturbations.

As shown in FIG. 5, multiple vibs 530, 540 may be separated by a substrate isolator 570. In this manner, vib1 530 solely effects the coupling 501 of element1 510 to a body surface 12 and, likewise, vib2 540 solely effects the coupling 502 of element2 520 to a body surface 12. Multiple isolated vibs 530, 540 advantageously allow each vib 530, 540 output to be adapted or otherwise suited to a particular element 510, 520, both in terms of amplitude and frequency. In an embodiment, the isolator 570 is a material that significantly attenuates mechanical/acoustical waves at the vib frequency or frequencies.

Also shown in FIG. 5, a monitor (not shown) outputs controls 562 to the sensor 500 and receives signals 562 from the sensor 500. The interface 560 communicates element signals 512, 522 to the monitor in response to drive controls 512, 522 to the elements 510, 520. The interface 560 also communicates vibration (vib) controls 564 to the vibs 530, 540. The vibs 530, 540 respond to the vib controls 564 so as to generate a modulation to their respect couplings 501, 502.

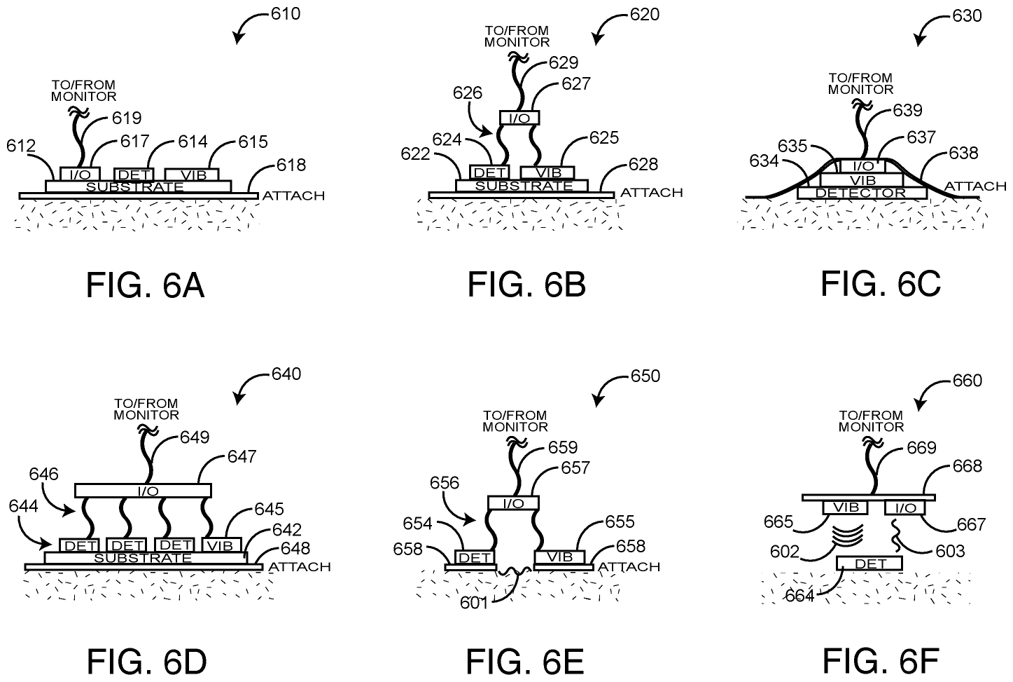

FIGS. 6A-F illustrate various modulated physiological sensor configurations. As shown in FIG. 6A, an integrated sensor embodiment 610 has a substrate 612, a detector 614, a vibration element (vib) 615, I/O (input/output) 617, an attachment 618 and electrical communication 619 to a monitor or similar device (not shown). The substrate 612 mounts the detector 614, vib 615 and I/O 617. In an embodiment, the substrate 612 also provides electrical trace interconnects between the I/O and both the detector 614 and vib 615. The I/O 617 transmits/receives sensor signals/controls and, in particular, drive to the vib 615 and signals from the detector 614. The attachment 618 adheres the substrate 612 and mounted components 614-617 to a body surface. In an embodiment, the detector 614 is mounted through the substrate 612 so as to couple directly to a body surface or via the attachment 618. The vib 615 advantageously modulates the coupling of the detector 614 to the body surface via the substrate 612 on which the detector 614 and vib 615 are co-mounted.

As shown in FIG. 6B, a semi-integrated sensor embodiment 620 has a substrate 622, a detector 624, a vib 625, I/O 627, an attachment 628 and electrical communication 629 to/from a monitor or other control or display device. The semi-integrated sensor embodiment 620 is similar to the integrated sensor embodiment 610 except that the I/O 627 is external to the sensor 620 and may be mounted in the monitor (not shown) or in a pod (not shown) between the sensor 620 and monitor. The I/O 627 is in electrical communications 626 with the detector 624 and vib 625, such as via cabling or other interconnect technology. The I/O 627 is also in electrical communications 629 with a monitor.

As shown in FIG. 6C, a substrate-less sensor embodiment 630 has a detector 634, a vib 635, I/O 637, an attachment 638 and electrical communications 639, which transmits signals and controls between the I/O 637 and a monitor or similar device (not shown). In this embodiment, the detector 634 or more specifically the detector package, such as a chip carrier, substitutes for a substrate. Accordingly, the vib 635 and I/O 637 are mounted within or on or otherwise directly coupled to the detector 634 package so that the detector 634 package is directly coupled to the body surface and held in place with the attachment 638. In an embodiment, the attachment 638 is simply an adhesive layer on the detector 634 package.

As shown in FIG. 6D, a sensor array embodiment 640 has a substrate 642, multiple detectors 644, a vib 645, I/O 647, an attachment 648 and electrical communication 649. The sensor array embodiment 640 is similar to the semi-integrated embodiment 620 (FIG. 6B) except for the multiple detectors 644. The detectors 644 may be all the same device type (mechanical, electrical, acoustical, etc.), all different or a mixture of one or more sub-arrays of the same device type with one or more different device types. Advantageously, multiple detectors 644 on a single substrate 642 provide an array of like sensors for increased detection capability or for directional sensing capability, such as determining the source of a body sound. Advantageously, multiple detectors 644 on a single substrate 642 provide an array of different detectors in a single sensor package for simultaneous detection and analyses of multiple types or kinds of physiological responses to the same or different external or internal perturbations. Advantageously, a mix of detectors and transmitters (not shown), such as one or more LEDs and one or more photodiode detectors, provide active sensing capabilities, such as illuminating and analyzing arterial (pulsatile) blood flow. Advantageously, one or more vibs 645 may provide both modulation and an active pulse for, say, analyzing non-pulsatile (venous) blood flow, as but one example.

As shown in FIG. 6E, a non-integrated sensor embodiment 650 has a detector 654, a vib 655 and attachments 658. The detector 654 and vib 655 are separately attached 658 to a body surface. The I/O 657 is in electrical communications 656 with the detector 654 and vib 655, such as via cabling or other interconnect technology, including wireless. Further, the I/O 657 is external to the sensor 650 and may be mounted in the monitor (not shown) or in a pod (not shown) between the sensor 650 and monitor with electrical communications 659 between the I/O 657 and the monitor. Advantageously, the vib 655 is attached to the body surface in close proximity to the detector 654 so that surface waves 601 generated by the vib in the body modulate the coupling between the detector 654 and the body surface.

As shown in FIG. 6F, a remote sensor embodiment 660 has a detector 664 and a modulation module 665. The modulation module 668 has a vib 665 and I/O 667. Advantageously, the vib 665 remotely modulates the detector 664 when brought into proximity to the detector 664. In particular, the vib 665 generates an acoustic wave 602 that vibrates the detector so as to modulate the detector coupling to the body surface. In particular, the acoustic wave 602 propagates through media intervening between the vib 665 and the detector 664. That media may be an air gap when the module 668 is positioned immediately over the detector 664 or the media may be tissue when the module 668 is positioned immediately over or on the body surface proximate the detector 664.

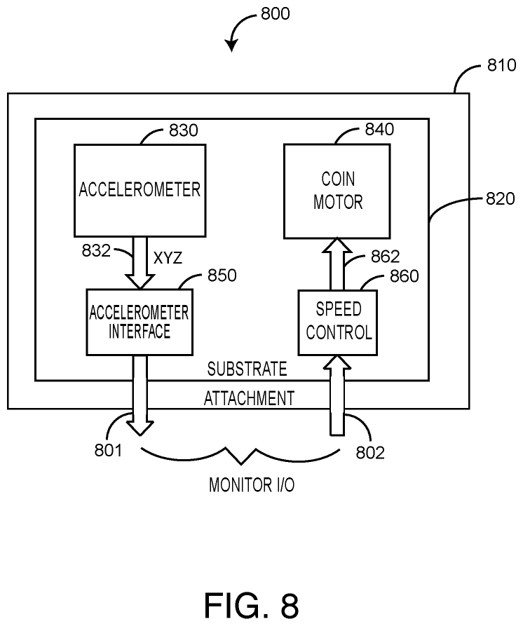

FIG. 7 generally illustrates a modulated physiological sensor 700 embodiment having an accelerometer 710 and a vibration element (vib) 720 mounted on a common substrate 730. An attachment (not shown) adheres or otherwise couples the substrate 730 to a body surface 12. The accelerometer 710 has three outputs 712 responsive to accelerations in three dimensions (x, y, z) advantageously enabling the sensor 700 to detect both the amplitude, direction and/or type of propagations (translational 85, 87 and longitudinal 86, 88) and whether the propagations are body waves 85, 86 or surface waves 87. The vib 720 mechanically modulates the coupling of the substrate 730 and, accordingly, the coupling of the accelerometer 710 to the body surface 12. The vib 720 frequency is selected to be substantially higher than the frequency of the propagations 85-88. As such, the accelerometer x, y and z outputs 712 are each amplitude modulated (AM) representations of the propagations 85-87. Advantageously, the modulated coupling substantially amplifies the propagations due to a peak AC coupling occurring once every cycle of the vib. That peak AC coupling is substantially greater than can be practically achieved with any static coupling of the accelerometer to the body surface 12. Accordingly, very low amplitude propagations can be detected and measured to yield physiological parameters. See, for example, a respiration rate sensor described with respect to FIGS. 8-10, below.

FIG. 8 is a detailed block diagram of a vibration-modulated physiological sensor 800 embodiment. The sensor 800 has an attachment 810, a substrate 820, an accelerometer 830, a coin motor 840 that generates vibration modulation, an accelerometer interface 850, a speed control 860 and monitor inputs/outputs (I/O) 801, 802. In an embodiment, the accelerometer 830 is an LIS352AX±2 g full scale, analog output, 3-axis (X, Y and Z) linear accelerometer available from STMicroelectronics, Geneva, Switzerland. In an embodiment, the coin motor 840 is a 10 mm coin motor 310-101 available from Precision Microdrives Ltd., London, UK. In an embodiment, the substrate 820 is a circuit board material that mechanically mounts and electrically interconnects the accelerometer 830, the coin motor 840, the accelerometer interface 850 and the speed control 860. In an embodiment, the attachment 810 is a sticky tape that mounts the sensor 800 to a body surface of a living being. In an embodiment, the monitor I/O 802 to the speed control is via a I²C bus. In an embodiment, the monitor I/O 801 to the accelerometer 830 includes a multiplexer control input to the accelerometer 830 to select one of the X, Y and Z axis for the accelerometer output 832 to the monitor. In another embodiment, all of X, Y and Z axes are simultaneously provided on the accelerometer output 832.

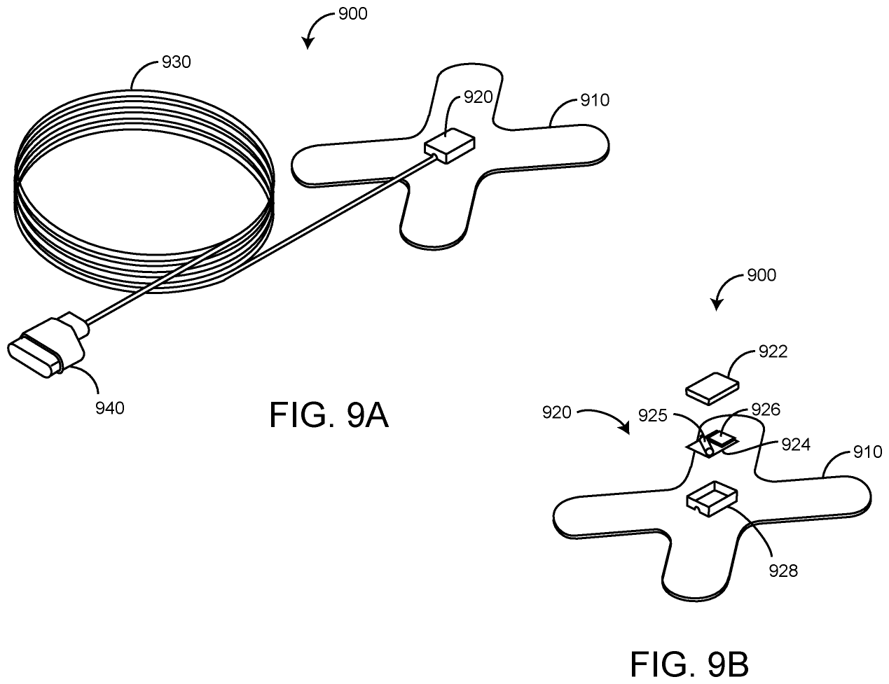

FIGS. 9A-B are assembled and exploded illustrations, respectively, of a vibration-modulated (vib) physiological sensor embodiment 900 that can be attached to a skin surface proximate various parts of a person's body, such as the chest, ribs, stomach, waist, arms or back so as to, for example, determine respiration-related parameters. In another embodiment, a modulated physiological sensor 900 may have an optical sensor (emitter and detector) combined with the accelerometer and vib. In this manner, the sensor can generate physiological measurements of pulsatile blood flow for blood constituent analysis, physiological measurements of non-pulsatile (venous) blood flow artificially pulsed by the vib and respiration measurements based upon either or both of pleth-modulated optical sensor waveforms and vib-modulated mechanical (accelerometer) waveforms.

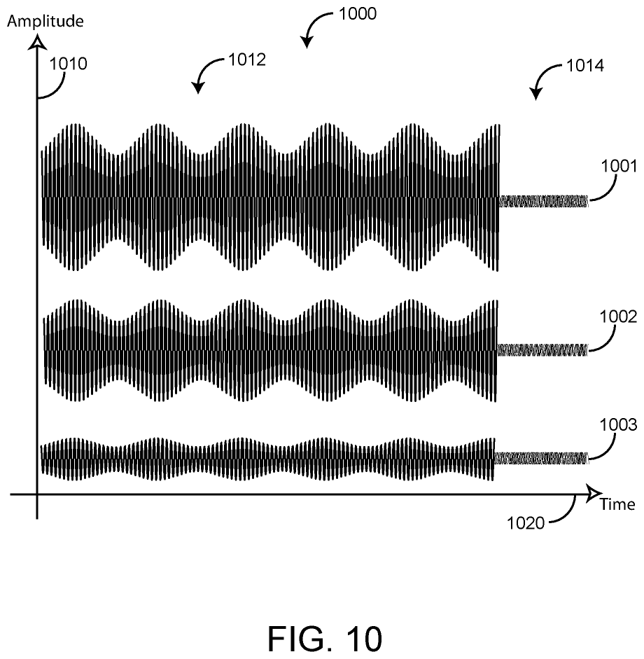

FIG. 10 is a vibration-accelerometer physiological sensor output 1000 illustrating three-axis respiration envelope amplitudes 1010 versus time 1020. The vibration continuously modifies the coupling of the accelerometer to the skin, which effectively multiples the measured acceleration due to respiration by that due to the vibration. This yields AM modulation waveforms 1001-1003 that display a (greatly magnified) respiration envelope. This effect is amply illustrated in comparing the difference in the accelerator response when the vibration (coupling modulator) is turned on 1012 and off 1014.

There are various applications for a modulated physiological sensor, as described above. A chest mounted sensor could monitor for sleep apnea at home, as well as in the hospital for patients receiving narcotics in the general wards. An abdomen-mounted sensor could monitor bowel sounds to give a quantifiable measurement to peristalsis. A dual sensor configuration, with one sensor mounted on the upper part of the abdomen and one on the lower part, is used for diagnosing bowel obstruction, small bowel volvulus or intussusception. A sensor mounted over the radial artery would yield a semi-continuous blood pressure measurement. Another configuration is a screening tool for sub-clinical stenosis of major vessels. For example, rather than placing a stethoscope over the carotid arteries or the abdomen to listen to flow through the aorta, a modulated sensor could give a more quantifiable measurement of stenosis, one level better than auscultation but one level below imaging. Another application is the differential diagnosis of heart murmurs aided by noise cancellation of breathing and other mechanical movements so as to distinguish distinctive murmur patterns (e.g. crescendo/decrescendo).

A modulated physiological sensor has been disclosed in detail in connection with various embodiments. These embodiments are disclosed by way of examples only and are not to limit the scope of the claims that follow. One of ordinary skill in art will appreciate many variations and modifications.

1.-20. (canceled)

21. A modulated physiological sensor comprising:
an accelerometer configured to generate a raw physiological signal responsive to a physiological process corresponding to a user, wherein the accelerometer is configured to be coupled to a skin surface of the user; and
a modulator configured to introduce an external signal separate from the physiological signal to modulate the physiological signal, wherein the modulated physiological signal improves a measurement of a physiological parameter compared to the raw physiological signal.

22. The modulated physiological sensor according to claim 21 wherein the modulator is a vibration element that mechanically accentuates the coupling of the accelerometer to the skin surface.

23. The modulated physiological sensor according to claim 22 further comprising a substrate that co-mounts the accelerometer and the vibration element.

24. The modulated physiological sensor according to claim 23 further comprising an attachment that releasably affixes the substrate, accelerometer and vibration element to the skin surface.

25. The modulated physiological sensor according to claim 24, further comprising an optical sensor.

26. The modulated physiological sensor according to claim 25 wherein the vibration element is a coin motor.

27. The modulated physiological sensor according to claim 26 wherein the substrate is a circuit board that mechanically mounts and electrically interconnects the accelerometer and the coin motor.

28. The modulated physiological sensor according to claim 27 wherein the attachment is a tape having a sticky side that attaches to the skin surface and a housing side that encloses the circuit board.
